(19) United States
(12) Patent Application Publication
(10) Pub. No.: US 2023/0277557 A1
(43) Pub. Date: Sep. 7, 2023

(54) PHARMACEUTICAL KITS AND THEIR USE FOR TREATING DRY EYE DISEASE

(71) Applicant: SURFACE OPHTHALMICS, INC., Pleasanton, CA (US)

(72) Inventors: Perry Sternberg, Mountain Lakes, NJ (US); Kamran Hosseini, Pleasanton, CA (US); Richard L. Lindstrom, Wayzata, MN (US)

(21) Appl. No.: 18/131,841

(22) Filed: Apr. 6, 2023

Related U.S. Application Data
(63) Continuation-in-part of application No. 18/100,505, filed on Jan. 23, 2023.
Application No. 18/100,505 is a continuation-in-part of application No. 17/582,627, filed on Jan. 24, 2022.
(63) Continuation-in-part of application No. PCT/US2021/053552, filed on Oct. 5, 2021.
Application No. 18/100,505 is a continuation-in-part of application No. 17/418,813, filed on Jun. 26, 2021.
Application No. 17/582,627 is a continuation-in-part of application No. 16/650,071, filed on Mar. 24, 2020.
(60) Provisional application No. 63/088,850, filed on Oct. 7, 2020.
(60) Provisional application No. 62/562,809, filed on Sep. 25, 2017.
(60) Provisional application No. 62/785,312, filed on Dec. 27, 2018.
(60) Provisional application No. 63/317,770, filed on Mar. 8, 2022.
(60) Provisional application No. 63/402,704, filed on Aug. 31, 2022.
(60) Provisional application No. 63/415,419, filed on Oct. 12, 2022.

(51) Int. Cl. A61K 31/573; A61K 45/06; A61K 9/00

(52) U.S. Cl. CPC A61K 31/573; A61K 9/0048; A61K 45/06

(57) ABSTRACT

Pharmaceutical kits and use of the kits in the treatment of conditions of the eye, such as dry eye disease, which includes a first composition including a corticosteroid, an immunosuppressant or both a corticosteroid and an immunosuppressant in a pharmaceutically acceptable carrier configured for administration to a subject’s eyes; a second composition including a corticosteroid in a pharmaceutically acceptable carrier configured for administration to a subject’s eyes, where the second composition has a higher concentration of corticosteroid than the first composition; and optionally a lubricating formulation that is pharmaceutical-free. Treatment of dry eye disease includes administering topically to the eyes of a subject suffering from chronic dry eye disease the first composition over a treatment period; and if the subject suffers from an episodic flare up during the treatment period, administering the second composition to substantially alleviate the flare-up.

### CROSS REFERENCE TO RELATED APPLICATIONS

This is a continuation in part of international patent application no. PCT/US2021/053552 filed Oct. 5, 2021, which claims priority to U.S. provisional Pat. application no. 63/088,850, filed Oct. 7, 2020; the entire content of each application referenced in this paragraph is herein incorporated by reference in its entirety.

This is also a continuation in part of U.S. Pat. application no. 18/100,505, filed Jan. 23, 2023, which is a continuation in part of U.S. Pat. application no. 17/582,627, filed Jan. 24, 2022, which is a continuation in part of U.S. Pat. application no. 16/650,071, filed Mar. 24, 2020, which is a U.S. national phase of international patent application no PCT/US2018/052158, filed Sep. 21, 2018, now expired, which claims priority to U.S. provisional application no. 62/562,809, filed Sep. 25, 2017, now expired; the entire content of each application referenced in this paragraph is herein incorporated by reference in its entirety.

U.S. Pat. application no. 18/100,505 is also a continuation in part of U.S. Pat. application no. 17/418,813, which is a U.S. national phase application of international patent application no. PCT/US2019/064371, filed Dec. 4, 2019, now expired, which claims priority to U.S. provisional application no. 62/785,312, filed Dec. 27, 2018, now expired; the entire content of each application referenced in this paragraph is herein incorporated by reference in its entirety.

U.S. Pat. application no. 18/100,505 also claims priority to U.S. provisional Pat. application no. 63/317,770, filed Mar. 8, 2022, now expired, and U.S. provisional Pat. application no. 63/402,704, filed Aug. 31, 2022, and U.S. provisional Pat. application no. 63/415,419, filed Oct. 12, 2022; the entire content of each application referenced in this paragraph is herein incorporated by reference in its entirety

### TECHNICAL FIELD

The invention relates generally to the field of ophthalmology and more specifically to pharmaceutical kits and their use for treating, mitigating, and/or preventing ocular surface disease, such as dry eye disease in mammals.

### BACKGROUND

Ocular surface disease, such as dry eye disease, is an ophthalmic condition that manifests itself in symptoms of discomfort and visual disturbance as a result of decreased tear production, and is characterized by a dysfunction of one or more components of the tear film, the latter being stable in the absence of this disease. Tear deficiency may be caused by poor production of tears as a result of age, hormonal changes, various autoimmune diseases, and other factors, and may also be a side effect of certain medications, such as beta-blockers, antidepressants, antihistamines, etc. However, normal stable condition of the tear film resulting in normal tear secretion is important for the lubrication and maintenance of the refractive surface of the eye.

Ocular surface disease may afflict an individual and vision may be substantially impaired with varying degrees of severity, ranging from burning sensation, a feeling of dryness and persistent irritation up to substantial impairment of vision in more severe cases. Therefore, a variety of approaches have been developed for treatment and therapy of such diseases. Typically, the majority of patients with an ocular surface disease are prescribed or recommended artificial tears. Other methods and devices that are also often recommended include scrubs, drops, inserts, plugs or lid compresses. These products typically include immunologic agents, autologous compounded serum, mucin producing agents and/or lubricants. While some such remedies do exist, and may provide some relief in some cases, in many other instances they are insufficient or too expensive.

Another challenge with treating dry eye disease is that the effects can vary drastically from day to day. For example, almost every patient, independent of therapy, will have a flare up, which causes extreme discomfort. Therefore, a more comprehensive approach for the treatment of dry eye disease would include addressing the underlying chronic conditions as wells as flare ups.

Accordingly, there remains a need for the treatment of dry eye disease that addresses both the chronic dry eye disease as well as episodic flare ups.

### SUMMARY OF THE INVENTION

The present disclosure addresses the above challenges and provides related benefits. In particular, the invention provides pharmaceutical kits formulated for treating or preventing conditions of the eye, and in particular ocular surface disease, such as dry eye disease. Among the improvements offered by the invention include kits that treat both chronic dry eye disease and acute dry eye disease, which is characterized by episodic flare ups that occur in subjects suffering from chronic dry eye disease. An exemplary kit includes a first composition including a corticosteroid, an immunosuppressant, or both a corticosteroid and an immunosuppressant in a pharmaceutically acceptable carrier configured for administration to a subject’s eyes; and a second composition including a corticosteroid in a pharmaceutically acceptable carrier configured for administration to a subject’s eyes, where the second composition is different than the first, such as by having a different corticosteroid or a higher concentration of a same corticosteroid than the first composition. The kit can also include a lubricating formulation that is pharmaceutical-free.

The kits can be used for the treatment of dry eye disease, where the first composition is configured for treating chronic dry eye disease and the second composition is configured for treating acute dry eye disease or episodic flare ups.

Preferably, kits are configured so that the first composition and second composition are each provided in a plurality of unit dose containers, where there is a different number of unit dose containers of the first composition compared to the second composition in the kit.

As to the first composition, exemplary corticosteroids include a betamethasone, betamethasone acetate, betamethasone sodium phosphate, loteprednol, a triamcinolone, triamcinolone acetonide, triamcinolone diacetate, triamcinolone benetonide, triamcinolone furetonide, triamcinolone hexacetonide, dexamethasone, fluorometholone, fluocinolone acetonide, prednisone, prednisolone, methylprednisone, corticol, cortisone, fluorocortisone, deoxycorticosterone acetate, aldosterone, and budesonide. A preferred corticosteroid is betamethasone sodium phosphate. Exemplary immunosuppressants include mycophenolic acid, mycophenolate sodium, mycophenolate mofetil, tacrolimus, cyclosporine, or pharmaceutically acceptable salts or derivatives thereof. A preferred immunosuppressant is mycophenolic acid or a pharmaceutically acceptable salt thereof (e.g. mycophenolate sodium). In some embodiments, the first composition also includes chondroitin sulfate.

As to the second composition, exemplary corticosteroids include a betamethasone, betamethasone acetate, betamethasone sodium phosphate, loteprednol, triamcinolone, triamcinolone acetonide, triamcinolone diacetate, triamcinolone benetonide, triamcinolone furetonide, triamcinolone hexacetonide, dexamethasone, fluorometholone, fluocinolone acetonide, prednisone, prednisolone, methylprednisone, corticol, cortisone, fluorocortisone, deoxycorticosterone acetate, aldosterone, and budesonide. In some embodiments, the second composition includes a different corticosteroid than the first composition. In other embodiments, the second composition includes a same corticosteroid as the first composition but at a higher concentration in the second composition. In other embodiments, the first composition is steroid free and the second composition contains a corticosteroid. A preferred corticosteroid is betamethasone sodium phosphate. The second composition can also include chondroitin sulfate.

The optional lubricating formulation can be substantially as provided in, as of the filing date of this application, REFRESH, THERASENSE, SOOTHE, and HYPOTEARS or any other tear lubricant. However a preferred lubricating formulation includes Sodium Thiosulfate Pentahydrate at 0.3 %wt/wt; Sodium Chondroitin Sulfate at 0.25% wt/wt; Dextran-70 at 0.25% wt/wt; Edetate Disodium Dihydrate at 0.1 % wt/wt; Poloxamer 407 at 0.2% wt/wt; Glycerin at 0.2% wt/wt; Potassium Chloride at 0.03% wt/wt; Sodium Phosphate Dibasic Anhydrous at 0.9% wt/wt; Sodium Phosphate Monobasic Anhydrous at 0.18% wt/wt; Sodium hydroxide at Q.S. to adjust pH 7.2; Hydrochloric Acid, 37% at Q.S. to adjust pH 7.2; and Sterile Water Q.S. to 100%.

Among beneficial uses of the kits include a method of treating dry eye disease (DED) in a subject, which includes providing a kit having a plurality of dose containers of each of the first composition and the second composition; administering to the subject suffering from chronic dry eye disease a therapeutically effective amount of the first composition over a treatment period; and if the subject suffers from an episodic flare up, administering the second composition to the subject to substantially alleviate the flare up. Once the episodic flare up is substantially alleviated, the method can include continuing to administer the first composition until another episodic flare up occurs.

### DETAILED DESCRIPTION OF PREFERRED EMBODIMENTS

Unless specific definitions are provided, the nomenclatures utilized in connection with, and the laboratory procedures and techniques of analytical chemistry, synthetic organic and inorganic chemistry described herein, are those known in the art. Standard chemical symbols are used interchangeably with the full names represented by such symbols. Thus, for example, the terms “hydrogen” and “H” are understood to have identical meaning. Standard techniques may be used for chemical syntheses, chemical analyses, formulating compositions and testing them. The foregoing techniques and procedures can be generally performed according to conventional methods well known in the art.

It is to be understood that both the foregoing general description and the following detailed description are exemplary and explanatory only and are not restrictive of the invention claimed. As used herein, the use of the singular includes the plural unless specifically stated otherwise. The section headings used herein are for organizational purposes only and are not to be construed as limiting the subject matter described.

As used herein, “or” means “and/or” unless stated otherwise. Furthermore, use of the term “including” as well as other forms, such as “includes,” and “included,” is not limiting. The phrase “consisting of” excludes any element, step, or ingredient not specified in the claim. The phrase “consisting essentially of” limits the scope of a claim to the specified materials or steps and those that do not materially affect the basic and novel characteristics of the claimed invention. The present disclosure contemplates embodiments of the invention, compositions, and methods corresponding to the scope of each of these phrases

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

The term “salt” refers to an ionic compound which is a product of the neutralization reaction of an acid and a base.

The terms “solvate” and “hydrate” are used herein to indicate that a compound or a substance is physically or chemically associated with a solvent for “solvates” such as water (for “hydrates”).

The term “mycophenolic acid” or “MPA” refers to the compound having the IUPAC name 6-(4-hydroxy-6-methoxy-7-methyl-3-oxo-1H-2-benzofuran-5-yl)-4-methylhex-4-enoic acid and the following chemical structure:

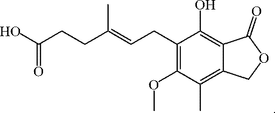

The term “cyclosporine” refers to the compound having the IUPAC name (3S,6S,9S,12R,15S,18S,21S,24S,30S,33S)-30-ethyl-33-[(E,1R,2R)-1-hydroxy-2-methylhex-4-enyl]-1,4,7,10,12,15,19,25,28-nonamethyl-6,9,18,24-tetrakis(2-methylpropyl)-3,21-di(propan-2-yl)-1,4,7,10,13,16,19,22,25,28,31-undecazacyclotritriacontane-2,5,8,11,14,17,20,23,26,29,32-undecone and the following chemical structure:

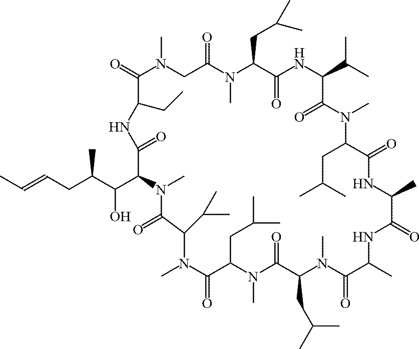 [SMILES: C/C=C/C[C@@H](C)C(O)[C@H]1C(=O)NC(CC)C(=O)N(C)CC(=O)N(C)[C@@H](CC(C)C)C(=O)N[C@@H](C(C)C)C(=O)N(C)C(CC(C)C)C(=O)N[C@@H](C)C(=O)NC(C)C(=O)N(C)[C@@H](CC(C)C)C(=O)N(C)C(CC(C)C)C(=O)N(C)C(C(C)C)C(=O)N1C]

The term “corticosteroid” refers to any steroid hormone, both produced synthetically and obtained from the adrenal cortex of vertebrates (inclusive of both glucocorticoids and mineralocorticoids) and belonging to a sub-genus of steroids that are derivatives of corticosterone, the latter having the chemical structure:

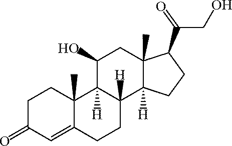

The term “tacrolimus,” also known as fujimycin or FK506, refers to an compound having the IUPAC name (-)-(3S,4R,5S,8R,9E,12S,14S,15R,16S,18R,26aS)-8-allyl-5,6,8,11,12,13,14,15,16,17,18,19,24,25,26,26a-hexadecahydro-5,19-dihydroxy-3-1{(E)-2-[(1R,3R,4R)-4-hydroxy-3-methylcyclohexyl]-1-methylvinyl}-14,16-dimethoxy-4,10,12,18-tetramethyl-15,19-epoxy-3H-pyrido[2,1-c][1,4]oxaazacyclotricosane-1,7,20,21(4H,23H)-tetrone, having the following chemical structure:

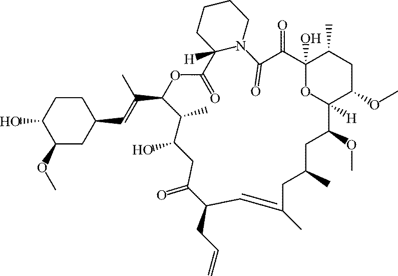 [SMILES: C=CC[C@@H]1/C=C(\C)C[C@H](C)C[C@H](OC)[C@H]2O[C@@](O)(C(=O)C(=O)N3CCCC[C@H]3C(=O)O[C@H](/C(C)=C/[C@@H]3CC[C@@H](O)[C@H](OC)C3)[C@H](C)[C@@H](O)CC1=O)[C@H](C)C[C@@H]2OC]

The term “glycosaminoglycan” refers to any unbranched polysaccharide having a repeating disaccharide unit.

The term “deturgescent agent” refers to a compound that is capable of maintaining the stroma of the cornea of the eye in a state of relative dehydration to an extent necessary to ensure the transparency of the cornea.

In various embodiments of the invention, the acceptable surfactant or solubilizing and suspending agent may be any of non-ionic polyoxyethlene-polyoxypropylene block copolymers, optionally partially cross-linked polyacrylates, polyoxyethylene sorbitan monolaurates, polyoxyethylene sorbitan monopalmitates, polyoxyethylene sorbitan monostearates, polyoxyethylene sorbitan monooleates, glyceryl distearate, triglycerol monooleate, and combinations thereof.

The term “ocular surface disease” (including “dry eye” or “dry eye syndrome” or “dry eye disease”) is defined as one or several conditions associated with, or caused by, either decreased or insufficient tear production or increased or excessive tear film evaporation, or both, and often characterized by redness, itching, and burning of the eye. An ocular surface disease is further defined as being inclusive of keratoconjunctivitis sicca, episodic dry eye disease, recalcitrant dry eye disease, age-related dry eye, neurotrophic ocular surface disease, meibomian gland disease and blepharitis. Symptoms associated with ocular surface disease and more specifically dry eye disease that are alleviated at least in part by the kits and methods herein, include stinging or burning sensation, excessive tearing, sensitivity to light, sandy or gritty sensation on the eye, episodes of blurred vision, eye fatigue, and eye redness. The term “chronic dry eye disease” refers to dry eye disease that persists over a long time or is consistently recurring. The term “episodic dry eye disease” refers to dry eye disease that occurs at regular or irregular intervals, which are typically characterized by greater discomfort than an underlying chronic dry eye disease.

The term “pharmaceutical composition” is defined as a substance that is subject to regulation as a drug, medicine or a controlled substance by the United States Food and Drug Administration (FDA) or foreign equivalent. A “pharmaceutical composition” contains a chemical or a biological compound or substance, or a mixture or combination of two or more such compounds or substances, intended for use in the medical diagnosis, cure, treatment, or prevention of disease or pathology. “Pharmaceutical compositions” include corticosteroids and immunosuppressants.

The term “pharmaceutical-free” is defined as a composition that lacks a compound that is subject to regulation as a drug, medicine or a controlled substance by the United States Food and Drug Administration (FDA) or foreign equivalent. The term “pharmaceutical-free” means the composition lacks both a corticosteroid and an immunosuppressant. A pharmaceutical-free composition can include a lubricating formulation or wetting formulation for lubricating or wetting the eyes.

The term “therapeutically effective amount” is defined as the amount of the compound or pharmaceutical composition that will elicit the biological or medical response of a tissue, system, animal or human that is being sought by the researcher, medical doctor or other clinician. Thus, the term “therapeutically effective amount” is used herein to denote any amount of a formulation that causes a substantial improvement in a disease condition when applied to the affected areas repeatedly over a period of time. The amount will vary with the condition being treated, the stage of advancement of the condition, and the type and concentration of formulation applied. Moreover, a “therapeutically effective amount” will likely differ between the first composition and second composition as each is intended for use in treating a different severity of dry eye disease, namely, chronic or acute dry eye disease.

The term “about” when used in the context of the formulations means that unless stated otherwise, the amount is preferred but could also vary by up to +/- 10%.

The term “pharmaceutically acceptable,” when used in the context of a carrier, is defined as a carrier, whether diluent or excipient, that is compatible with the other ingredients of the formulation and not deleterious to the recipient thereof. The term “pharmaceutically acceptable carrier configured for administration to a subject’s eyes” means that the carrier is suitable for topical administration to the eyes of the recipient.

The terms “administration of a composition” or “administering a composition” are defined to include an act of providing a compound or pharmaceutical composition of the invention to the subject in need of treatment.

The term “subject,” as used herein, refers to any individual or patient to which the subject methods are performed. Generally, the subject is human, although as will be appreciated by those in the art, the subject may be an animal.

As used herein, “treatment” or “treating” means to administer a composition to a subject with an undesired condition. The condition can include a disease or disorder. “Prevention” or “preventing” means to administer a composition to a subject at risk for the condition. The condition can include a predisposition to a disease or disorder. The effect of the administration of the composition to the subject (either treating and/or preventing) can be, but is not limited to, the cessation of one or more symptoms of the condition, a reduction or prevention of one or more symptoms of the condition, a reduction in the severity of the condition, the complete ablation of the condition, a stabilization or delay of the development or progression of a particular event or characteristic, or minimization of the chances that a particular event or characteristic will occur.

Turning now to preferred embodiments of the disclosure, provided are kits and their use for the prevention and treatment of ocular surface disease. The kits are particularly useful for the treatment of dry eye disease. Still further, the kits are useful for the treating subjects suffering from chronic dry eye disease and/or suffering from or at risk of suffering from acute dry eye disease or episodic flare ups that can occur in subjects suffering from chronic dry eye disease. This is accomplished by way of a kit preconfigured for the treatment of dry eye disease that may vary in severity of symptoms over time. A preferred kit contains a first composition for treating chronic dry eye disease, and a second composition for treating acute episodic flare ups associated with dry eye disease. To this end, the kits are effective at relieving fluctuations in the severity of most, if not all of the symptoms associated with dry eye disease including, but not limited to, burning sensation in the eyes, eye itchiness, aching sensation in the eyes, eye heaviness, eye fatigue, eye soreness, and eye dryness. One of ordinary skill in the art to which the invention belongs will appreciate that the kits can be used when a subject is suffering from or is at risk of suffering from both chronic dry eye disease as well as episodic flare ups. As nonlimiting examples, the kit can be used after an eye surgery or after the eye is wounded, whether intentionally or unintentionally. Alternatively, or in addition, the kit can be used for rehabilitating the ocular surface before and/or after contact lens wear. As such, the treatment period for chronic dry eye disease therapy using the first composition can vary from days to weeks to months and more; whereas, the treatment of episodic flare ups using the second composition tends to occur instances where the subject temporarily suffers from heightened pain or discomfort. Thus, while treating the chronic dry eye disease using the first composition, when the subject suffers from heightened pain or discomfort the second composition may be temporarily administered to substantially alleviate this heightened pain or discomfort.

As will be described in more detail in the passages that follow, each composition within the kit is preferably delivered or administered to the eyes topically (e.g. via eye drops), though gels and ointments are also envisioned.

Turning now to the kit components, the kit includes a first composition in a pharmaceutically acceptable carrier formulated for treating chronic dry eye disease; a second composition that is different from the first and in a pharmaceutically acceptable carrier formulated for treating acute episodic flare ups of dry eye disease; and optionally a lubricating formulation for treating dry eye that is pharmaceutical-free. For clarity of disclosure, the second composition being different from the first does not exclude instances where both the first and second compositions include a same active ingredient, such as a corticosteroid, however in such instances the first and second compositions would have a different concentration of that same active ingredient. As a nonlimiting example the first composition may contain a corticosteroid at 0.01% wt/wt and the second composition a same corticosteroid at 0.02% wt/wt. When corticosteroids are provided in both the first and second compositions, typically the concentration will be higher in the second.

It is envisioned that in some embodiments, pharmacists will package the kits with first and second compositions according to the patients’ needs as prescribed by a physician. For example, a chronic dry eye disease therapy may include a prescribed number of unit doses of the first composition, while an acute therapy (for episodic flare ups), which may occur periodically over the course of the chronic therapy treatment, can include a prescribed number of unit doses of the second composition. It is expected that both the first and second compositions will be provided in the kits. This permits the kits to be assembled according to the needs of each subject. It is also envisioned that the kit can contain a number of pharmaceutical-free lubricant doses, such as for use when the subject’s eyes become generally dry. To this end, the pharmacist can be supplied with a plurality of dose containers for each of the first composition and second composition, then package the appropriate number of dose containers of each as prescribed. As a nonlimiting example, when providing a plurality of dose containers each container may contain between 0.1 mL and 1 mL, preferably .3 mL to .5 mL, so that each dose container (also called a unit dose container) contains a single dose. Thus, in preferred embodiments, the kit will contain more than one container for each composition; however, it is also envisioned the kit could contain a single container for at least one of the first composition, the second composition and a pharmaceutical-free lubricant.

Alternatively, the kits may be pre-packaged at the manufacturer with a specific number of unit dose containers for each of the chronic therapy, acute therapy, and lubricating solution. In such an embodiment, several different kits can be packaged with specific numbers of unit dose containers for chronic treatment (first composition), acute treatment (second composition) and optionally lubricant (lubricant solution), where each unit dose container contains a single unit dose. In such instances, the number of unit dose containers for the first composition (chronic treatment) will be greater than the number of unit doses for the second composition (acute/episodic treatment). Sample kit unit dose container numbers are illustrated in Table 1.

TABLE 1

Exemplary Pre-packaged Kit Configurations

| Kit Designation | Chronic Treatment Number of Unit Dose Containers^{∗} | Acute Treatment Number of Unit Dose Containers^{∗} | Lubrication Solution Number of Unit Dose Containers^{∗} |
|---|---|---|---|
| Group A | 180 | 60 | 60 |
| Group B | 90 | 30 | 30 |
| Group C | 180 | 30 | 30 |

^{∗}Each kit containing up to the maximize number of doses in each column

The containers, whether packaged by a pharmacy or manufacturer can be opaque when filled and can be wrapped with a label or metal foil to prevent the formulation from being exposed to substantial light. A container including a metal foil also advantageously reduces oxygen permeability from the atmosphere through the container. The containers are preferably of the type for single dose administration e.g., in a bottle, jar, ampoule, tube, envelope, container, unit dose container or vial.

In any event, administration instructions should be added, which include suggested frequency of administration. Without being bound by theory, it is likely that the first composition, which is for the treatment of chronic condition, will be administered regularly, such as 1 to 16 times a day (e.g., from 1 to 8 times per day, from 1 to 6 times per day, or from 1 to 4 times per day, 1-2 times per day), or more frequently, or less frequently; whereas the second composition for treating episodic flare ups will be administered less regularly, such as when corresponding symptoms (e.g. intense burning sensation in the eyes, heightened eye itchiness or aching, extreme dryness) are present.

It will be understood by those having ordinary skill in the art to which the invention belongs that the specific dose level and frequency of dosage for any particular patient may be varied and will depend upon a variety of factors including the activity of the specific compound employed, the metabolic stability and length of action of that compound, the age, body weight, general health, gender, diet, and the severity of the particular disease or condition being treated.

Turning now to the compositions formulated for treating chronic dry eye disease, in some embodiments the first composition described herein includes a corticosteroid. Preferably, the corticosteroid is selected from the group consisting of a betamethasone, betamethasone acetate, betamethasone sodium phosphate, loteprednol, triamcinolone, triamcinolone acetonide, triamcinolone diacetate, triamcinolone benetonide, triamcinolone furetonide, triamcinolone hexacetonide, dexamethasone, fluorometholone, fluocinolone acetonide, prednisone, prednisolone, methylprednisone, corticol, cortisone, fluorocortisone, deoxycorticosterone acetate, aldosterone, and budesonide. More preferably the first composition includes betamethasone sodium phosphate.

Alternatively, or in addition, the first composition can include an immunosuppressant. Preferably, the immunosuppressant includes, but is not limited to, mycophenolic acid, tacrolimus, cyclosporine, or any pharmaceutically acceptable salt or derivative thereof. More preferably, the immunosuppressant is selected from the group consisting of mycophenolate sodium, mycophenolate mofetil or mycophenolic acid. Most preferably the immunosuppressant is mycophenolic acid or a pharmaceutically acceptable salt thereof.

In some embodiments, the first composition, which is configured for treating the chronic condition, includes the corticosteroid but not the immunosuppressant. In other embodiments, the first composition includes the immunosuppressant but not the corticosteroid. However, in still other embodiments, the first composition includes both the corticosteroid and the immunosuppressant. In each of the embodiments, the composition can also include chondroitin sulfate.

The kits described herein have been developed from results, which found that the addition of a very low concentration of a corticosteroid (e.g., betamethasone sodium phosphate) to an immunosuppressant, such as mycophenolic acid, provides superior and/or synergistic efficacy compared to standard treatment protocols for the treatment of dry eye disease. In consideration of the very low concentration of corticosteroid incorporated into such treatments, no negative impact on safety has been found.

After still further extensive research, it was found that while betamethasone sodium phosphate and mycophenolic acid when used together provided a substantial improvement in the treatment of chronic dry eye disease, a still better treatment regimen could be developed and adapted for individual patients or different groups of patients, in which the chronic dry eye condition could be treated using a combined therapy of a corticosteroid and immunosuppressant while episodic flare ups, which tend to be temporary but more intense, could be temporarily treated using a composition that decreased the dosage of the immunosuppressant and increased the dosage of the corticosteroid.

To this end, a second composition, which is different from the first was developed. In particular, the second composition, which is configured for treating acute episodic flare ups of dry eye disease, can include a corticosteroid such as one or more of a betamethasone, betamethasone acetate, betamethasone sodium phosphate, loteprednol, triamcinolone, triamcinolone acetonide, triamcinolone diacetate, triamcinolone benetonide, triamcinolone furetonide, triamcinolone hexacetonide, dexamethasone, fluorometholone, fluocinolone acetonide, prednisone, prednisolone, methylprednisone, corticol, cortisone, fluorocortisone, deoxycorticosterone acetate, aldosterone, and budesonide. The second composition differs from the first composition in that the amount of corticosteroid in the second composition is generally greater than the first. Moreover, the immunosuppressant is generally not added when treating episodic flare ups. The result is a temporal use of a higher dose of corticosteroid in instances of flare ups, while using a lower dose of corticosteroid together with the immunosuppressant during more the prolonged treatment period.

In furtherance of the above, in a preferred embodiment, the first composition includes a betamethasone, namely, betamethasone sodium phosphate, and mycophenolic acid. As general guidance, the corticosteroid is typically provided between 0.001% wt/wt and 0.8% wt/wt. In a preferred embodiment that includes both betamethasone sodium phosphate and mycophenolic acid in the first composition, the betamethasone sodium phosphate can be provided in an amount such as 0.005% wt/wt to 0.05% wt/wt and the mycophenolic acid in a range of 0.05% wt/wt to 0.5% wt/wt. In some embodiments, the betamethasone is provided in the composition from 0.008 % wt/wt to 0.04 % wt/wt, from 0.009 % wt/wt to 0.04 % wt/wt, from 0.01 % wt/wt to 0.04 % wt/wt, from 0.02 % wt/wt to 0.04 % wt/wt, from 0.01% wt/wt to 0.03 wt/wt, from 0.01% wt/wt to 0.02% wt/wt, 0.02 % wt/wt, 0.03% wt/wt, or 0.04 % wt/wt. Higher dosages can also be used such as 0.05%, 0.06%, 0.07%, 0.08%, 0.09%, 0.1%, 0.125%, and 0.15%; however, these higher doses are not preferred especially for extended treatment over time. In some embodiments the mycophenolic acid is provided in the composition from 0.08 % wt/wt to 0.4 % wt/wt, from 0.09 % wt/wt to 0.4 % wt/wt, from 0.1 % wt/wt to 0.3 % wt/wt, from 0.2% wt/wt to 0.4 % wt/wt, 0.2 % wt/wt, 0.3 % wt/wt, or 0.4 % wt/wt. Mycophenolic acid may be provided from 0.05% w/w to 0.2% w/w, from 0.05% w/w to 0.1% w/w, from 0.1% w/w to 0.2% w/w, from 0.1% to 0.25% w/w, or from 0.1% to 0.3% w/w. Again, mycophenolic acid could be used at higher amounts, such as but not limited to 0.5%, 0.6%, 0.7%, 0.8%, 0.9% and 1% but these higher doses have been found less effective. Currently a preferred first composition contains 0.01% wt/wt betamethasone sodium phosphate and 0.1% w/w mycophenolic acid. Another preferred first composition contains 0.01% wt/wt betamethasone sodium phosphate and 0.2% wt/wt mycophenolic acid. Another preferred first composition contains 0.01% wt/wt betamethasone sodium phosphate and 0.3% wt/wt mycophenolic acid. Each of the above can be provided in the formulation shown in Table 2.

One of ordinary skill in the art to which the invention belongs will recognize that when using other corticosteroids the physician may choose to use a different amount compared to the above guidance for betamethasone. As a nonlimiting example, the formulation in Example 2, provides loteprednol at 0.2%. Thus, the skilled artisan may decide to use an amount of corticosteroid or immunosuppressant that is higher or lower that the preferred ranges stated above with respect to betamethasone and mycophenolic acid.

In some embodiments, the second composition includes a corticosteroid that differs from that of the first composition. However, in other embodiments, the second composition includes a same corticosteroid (e.g. betamethasone sodium phosphate) as the first composition but does not include the immunosuppressant. In such a configuration, the corticosteroid is intended for use temporally, such as during episodic flare ups and is generally provided at a higher dose than in the first composition. As a non-limiting example, the second composition can use a same corticosteroid as the first composition but at a dose that is up to twice or three times the dose of the same corticosteroid in the first composition, such as 10% higher, 20% higher, 30% higher, 40% higher, 50% higher, 60% higher, 70% higher, 80% higher, 90% higher, 100% higher, 110% higher, 120% higher, 130% higher, 140% higher, 150% higher, 160% higher, 170% higher, 180% higher, 190% higher, 200% higher or more. As a nonlimiting example, when using betamethasone sodium phosphate in the first composition at 0.04% wt/wt, it may be desirable to use betamethasone sodium phosphate in the second composition at 0.05% wt/wt, 0.06% wt/wt, 0.07% wt/wt, 0.08% wt/wt or more. As another example, a first composition may include betamethasone sodium phosphate at 0.01% wt/wt and the second composition may include betamethasone sodium phosphate at 0.02% wt/wt. Most preferably each is provided in the carrier disclosed in Table 2.

In each of the above embodiments, the first and second compositions also include a pharmaceutically acceptable carrier configured for administration to a subject’s eyes, which can be the same or different, such as but not limited to de-ionized water and/or a balanced salt solution. In various embodiments, the balanced salt solution used in the compositions of the invention includes potassium chloride, calcium chloride, magnesium chloride, sodium chloride, sodium acetate and sodium citrate. In some embodiments the pharmaceutically acceptable carrier is a same or substantially the same as a lubricating formulation that is also provided in the kit. In a particularly preferred embodiment, the lubricating formulation is provided substantially as set forth in Table 2, which can also form the pharmaceutically acceptable carrier for the first composition, the second composition or both.

TABLE 2

Exemplary Lubricating Formulation

| | | |
|---|---|---|
| Ingredient | Grade | Amount/Concentration (% Wt/Wt) |
| Sodium Thiosulfate Pentahydrate | USP | 0.3 |
| Sodium Chondroitin Sulfate | NC | 0.25 |
| Dextran-70 | USP | 0.25 |
| Edetate Disodium Dihydrate | USP | 0.1 |
| Poloxamer 407 | NF | 0.2 |
| Glycerin | USP | 0.2 |
| Potassium Chloride | USP | 0.03 |
| Sodium Phosphate Dibasic Anhydrous | USP | 0.9 |
| Sodium Phosphate Monobasic Anhydrous | USP | 0.18 |
| Sodium hydroxide | NF | Q.S. to adjust pH 7.2 |
| Hydrochloric Acid, 37% | NF | Q.S. to adjust pH 7.2 |
| Sterile Water for Injection | USP | Q.S. to 100% |

In other embodiments, the lubricating formulation, and optionally the pharmaceutically acceptable carrier, can be substantially as provided in REFRESH, THERATEARS, SOOTHE, or HYPOTEARS as of the filing date of the present application. Other lubricants or wetting solutions can also be used.

As general guidance, without limitation, and in view of the formulations herein, an exemplary kit for the treatment of conditions of the eye may be as follows: Chronic therapy: A formulation of a combination of 0.01% betamethasone sodium phosphate and 0.1%-0.3% mycophenolic acid packaged in 0.3 mL fill unit dose container with 180-unit dose containers. Acute therapy: A formulation of 0.02% -0.04% betamethasone sodium phosphate a with a 0.3 mL fill in 60-unit dose containers. Dry eye Lubricant: Formulation set forth in Table 2 with 60-unit dose containers with a 0.3 mL fill. Preferably, the pharmaceutically acceptable carrier for both the first and second composition is the formulation set fort in Table 2.

Another nonlimiting example of a kit for the treatment of conditions of the eye may be as follows: Chronic therapy: A formulation containing 0.1%-0.3% mycophenolic acid packaged in 0.3 mL fill unit dose container with 180-unit dose containers. Acute therapy: A formulation of 0.02% betamethasone sodium phosphate a with a 0.3 mL fill in 30-unit dose containers. Dry eye Lubricant: 30-unit dose containers of REFRESH the over the counter dry eye lubricant.

Another nonlimiting example of a kit for the treatment of conditions of the eye may be as follows: Chronic therapy: A formulation containing 0.01% betamethasone sodium phosphate packaged in 0.3 mL fill unit dose container with 180-unit dose containers. Acute therapy: A formulation of 0.02% betamethasone sodium phosphate a with a 0.3 mL fill in 30-unit dose containers. Dry eye Lubricant: 30-unit dose containers of REFRESH the over the counter dry eye lubricant.

Turning back to the first and second compositions more broadly, in some embodiments, the first and/or second compositions provided herein are provide with a lubricating agent, such as but not limited to glycerol or glycerin. Alternatively, or in addition thereto, the lubricating agent may include one or more secondary lubricating agents. Non-limiting examples of acceptable secondary lubricating agent(s) that may be so used include any of: polyvinylpyrrolidone, sorbitol, polyethylene glycol, hydroxypropyl methylcellulose, carboxymethylcellulose, and polyvinyl acetate. In various embodiments, the total content of the lubricant (regardless of the total number of lubricating agents used) expressed as the mass concentration may be between about 0.1 % wt/wt and about 5.0 % wt/wt, such as between about 1.0 % wt/wt and about 4.0 % wt/wt, for example, preferably about 1.0 % wt/wt.

In some embodiments, the first and/or second compositions provided herein are provided with at least one glycosaminoglycan. It can be theorized, without firm commitment to any particular or specific mechanism, that glycosaminoglycans may be useful in protecting endothelial and epithelial cells which are subject to exposure to trauma, and/or to promote the growth of such cells. Non-limiting examples of glycosaminoglycan(s) that may be used include: chondroitin, chondroitin sulfate, dermatan sulfate, dermatin sulfate, heparin sulfate, heparan sulfate, keratin sulfate, keratan sulfate, and hyaluronic acid. In various embodiments, the glycosaminoglycan that can be so used is chondroitin sulfate. The total content of the glycosaminoglycan(s) used with each composition, when present, expressed as the mass concentration may be from about 0.1 % wt/wt to about 5.0 % wt/wt, such as from about 0.1% wt/wt to about 0.3% wt/wt, from about 0.2 % wt/wt to about 4.0 % wt/wt, for example preferably about 0.25 % wt/wt.

In some embodiments, the first and/or second compositions provided herein are provided with at least one deturgescent agent. One non-limiting example of an acceptable deturgescent agent that may be so used is dextran sulfate. Non-limiting examples of other acceptable deturgescent agent(s) that may be used in addition to, or instead of, dextran sulfate include any of: dextran, sodium chloride, dextrose, and sucrose. While such deturgescent agents are typically used to provide dehydration for stroma of the cornea of the eye, it has been unexpectedly found that inclusion of at least one deturgescent agent may be beneficial for improving outcomes in the treatment of various surface ocular diseases such as dry eye diseases. The total content of the deturgescent agent(s) used with each composition expressed as the mass concentration may be between 0.1 % wt/wt and 5.0 % wt/wt, such as between 0.2 % wt/wt and 4.0 % wt/wt, for example preferably 0.25 % wt/wt.

In some embodiments, the first and/or second compositions provided herein are provided with one or more antioxidants selected from the group consisting of ascorbic acid derivatives such as ascorbic acid, erythorbic acid, and sodium ascorbate; thiol derivatives such as thioglycerol, cysteine, acetylcysteine, cystine, dithioerythreitol, dithiothreitol, glutathione; tocopherols; butylated hydroxyanisol (BHA); butylated hydroxytoluene (BHT); sulfurous acid salts such as sodium sulfate, sodium bisulfite, acetone sodium bisulfite, sodium metabisulfite, sodium sulfite, sodium formaldehyde sulfoxylate, and sodium thiosulfate; and nordihydroguaiaretic acid.

As mentioned above, in addition to the above-described components, each of the first and second compositions provided herein are provided with a carrier. In various embodiments, the carrier is pure de-ionized water and may further include a balanced salt solution, as is known to those having ordinary skill in the art. In yet other embodiments, the carrier may, in addition to water and/or the balanced salt solution, further optionally contain one or more pharmaceutically acceptable excipient(s) or surfactants. In various embodiments total content of the excipient or surfactant portion for each composition (regardless of the number of excipients or surfactants used) expressed as the mass concentration may be between 0.1 % wt/wt and 5.0 % wt/wt, such as between 0.1 % wt/wt and 4.0 % wt/wt, between 0.2 % wt/wt and 4.0 % wt/wt, between 0.3 % wt/wt and 3.0 % wt/wt, between 0.4 % wt/wt and 2.0 % wt/wt, or preferably, between 0.5 % wt/wt and 1.0 % wt/wt.

In various embodiments, the excipient or surfactant may be a non-ionic polyoxyethlene-polyoxypropylene block copolymer having the following general structure:

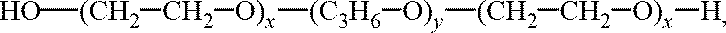

wherein x is an integer having the value of at least 8 and y is an integer having the value of at least 38.

One non-limiting example of a specific non-ionic polyoxyethlene-polyoxypropylene block copolymer that can be used as a excipient or surfactant with the first and/or second compositions of the instant invention is the product known under the trade names PLOXAMAR 407 and PLURONIC F-127 (poly(ethylene glycol)-block-poly(propylene glycol)-block-poly(ethylene glycol)), with the molecular weight of the polyoxypropylene portion of about 4,000 Daltons, about a 70% polyoxyethylene content, the overall molecular weight of between about 9,840 Daltons and about 14,600 Daltons. Another non-limiting example of a non-ionic poly(oxyethlene-co-oxypropylene) block copolymer that can be used is the product known under the trade name Poloxamer 188 (P188), which has an overall molecular weight of about 8400 Daltons. Each of which have the following chemical structure:

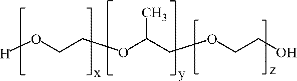

where x and z = 80 and y = 27 for P188; and where x and z = 101 and y = 56 for P407.

Another type of product that can be used in the excipient or surfactant portion of the first and/or second composition may be water-soluble methylcellulose and hydroxypropyl methylcellulose polymers, such as METHOCEL family of products, for example, a hydroxypropyl methylcellulose product METHOCEL E4M. The compositions may also contain a quantity of preservative(s) such as benzalkonium chloride, if desired.

Yet another type of product that can be used in the excipient or surfactant portion of the first and/or second composition may be a polycarbophil polymer product (i.e., a polymeric product based on polyacrylic acid cross-linked with divinyl glycol) which is available under a variety of trade names such as FIBERCON, EQUALACTIN, KONSYL FIVER, etc. If a polycarbophil product is used it may also be present as a part of mycophenolic acid solution, as mentioned above.

Additional exemplary excipients or surfactants that can be used herein include, but are not limited to, edetate disodium dihydrate (EDTA), polysorbate-80, sodium thiosulfate, sodium thiosulfate pentahydrate, sodium phosphate, sodium phosphate dibasic anhydrous, and sodium phosphate monobasic anhydrous.

Finally, each of the formulations for the first and second compositions will typically have an osmolarity between about 100 and about 500 milliosmoles per liter (mOsm/L), such as between about 150 mOsm/L and about 450 mOsm/L, for example, preferably between about 200 mOsm/L and about 400 mOsm/L. A tonicity modulating agent, such as sodium chloride, may also be used in the compositions.

As is known in the art, certain pharmaceutical compositions including mycophenolic acid or a salt thereof as an active ingredient in an aqueous medium with a reduced level of dissolved oxygen relative to saturation level are more stable than formulations with aqueous media having saturated levels of dissolved oxygen. Thus, preparing the pharmaceutical compositions described herein may optionally further include a step of removing dissolved oxygen from the formulation. The removal of dissolved oxygen from the composition can be carried out by any conventional means including applying a vacuum to the medium and/or inert atmosphere distillation, freeze-thaw degassing, use of degassing filters, etc., as described in U.S. Pub. No. 2017/006552, incorporated herein by reference. Thus, the process for preparing such compositions can further include packaging the composition in an opaque (e.g., amber) container to prevent exposure of the composition to a substantial amount of light and/or with an inert atmosphere. In various embodiments, the process can include purging the empty container with an inert gas, e.g., N₂, before filling and subsequently filling the container with the pharmaceutical composition.

The following examples are provided to further elucidate the advantages and features of the present invention but are not intended to limit the scope of the invention. The example is for the illustrative purposes only. USP pharmaceutical grade products were used in preparing the formulations described below.

### Example 1

### Preparing an Immunosuppressant-Based Pharmaceutical Composition for Use in the Treatment of Chronic Dry Eye Disease

A pharmaceutical composition was prepared as described below. The following components were used in the amounts specified:
(a) about 0.321 g of mycophenolate sodium;
(b) about 0.05% cyclosporine;
(c) about 0.25 g of chondroitin sulfate (bovine);
(d) about 0.10 g of powdered edetate disodium dihydrate;
(e) about 0.2 g of PLURONIC F-127;
(f) about 1.0 mL of glycerol;
(g) about 0.2% polysorbate-80;
(h) about 0.125 g of METHOCEL E4M; and
(i) about 100 mL of balanced salt solution.

Mycophenolate sodium, chondroitin sulfate, edetate disodium dehydrate, polysorbate-80 and PLURONIC F-127 were combined with about 90% of the balanced salt solution and stirred until completely dissolved. With continued stirring, METHOCEL E4M was added followed by addition of glycerol. The pH of the solution was then adjusted to about 6.8-7.2 using sodium hydroxide solution and the remainder of the balanced salt solution was added.

This first step was then filtered through a 0.2 micron filter and placed in an ISO 5 environment with continued stirring. Using aseptic technique, sterile cyclosporine powder was then added to the sterilized solution prepared as described above. The final product was then filled aseptically into pre-sterilized dropper bottles or unit dose vials and sealed.

### Example 2

### Preparing a Combined Corticosteroid and Immunosuppressant-Based Pharmaceutical Composition For Use in the Treatment of Chronic Dry Eye Disease

A pharmaceutical composition was prepared as described below. The following components were used in the amounts specified:
(a) about 0.321 g of mycophenolate sodium powder;
(b) about 0.2 g of loteprednol etabonate;
(c) about 0.25 g of chondroitin sulfate (bovine);
(d) about 0.10 g of powdered edetate disodium dihydrate;
(e) about 0.2 g of PLURONIC F-127;
(f) about 1.0 mL of glycerol;
(g) about 0.1 g polysorbate-80;
(h) about 1 g of polyvinylpyrrolidone;
(i) about 0.125 g of METHOCEL E4M; and
(j) about 100 mL of balanced salt solution.

Mycophenolate sodium, chondroitin sulfate, edetate disodium dehydrate, polyvinylpirrolidone, polysorbate-80 and PLURONIC® F-127 were combined with about 90% of the balanced salt solution and stirred until completely dissolved. With continued stirring, METHOCEL E4M was added followed by addition of glycerol. The pH of the solution was then adjusted to about 5.6 to 5.8 using sodium hydroxide solution and the remainder of the balanced salt solution was added.

The resulting solution was then filtered through a 0.2 micron filter and placed in an ISO 5 environment with continued stirring. Using aseptic technique, sterile loteprednol etabonate powder was then added to the sterilized solution above. The final product was then filled aseptically into pre-sterilized dropper bottles or unit dose vials and sealed.

### Example 3

### Preparing a Combined Corticosteroid and Immunosuppressant-Based Pharmaceutical Composition For Use in the Treatment of Chronic Dry Eye Disease

A pharmaceutical composition was prepared as described below. The following components were used in the amounts specified:
(a) about 0.321 g of mycophenolate sodium powder;
(b) about 0.01 g of betamethasone sodium phosphate powder;
(c) about 0.25 g of chondroitin sulfate (bovine);
(d) about 0.25 of powdered dextran 70,000;
(e) about 0.30 g of powdered sodium thiosulfate pentahydrate;
(f) about 0.20 g of PLURONIC F-127;
(g) about 1.0 mL of glycerol;
(h) about 1.17 g of sodium phosphate dibasic anhydrous;
(i) about 0.4 g of sodium phosphate monobasic anhydrous;
(j) about 0.10 g of METHOCEL E4M;
(k) about 40 mL of balanced salt solution; and
(l) about 100 mL of sterile injectable water.

Chondroitin sulfate, dextran, sodium thiosulfate, and PLURONIC F-127 were combined with 90% of the balanced salt solution and about 90% of the water and stirred until completely dissolved followed by addition of glycerol with continued stirring. The pH of the solution was then adjusted to about 7.0 using sodium hydroxide solution before introducing mycophenolate sodium. A stock solution of 1% betamethasone was prepared by dissolving 1 gm of betamethasone sodium phosphate powder in 100 mL water and confirming the stock solution concentration by HPLC. 1ml of the 1% betamethasone stock solution was then added to the mixture with continued stirring.

With continued stirring, mycophenolate sodium was added slowly followed by addition of METHOCEL E4M, adjusting pH to about 7.3-7.4, and addition of the remainder of the water. The solution was then filtered through a 0.2 micron filter into sterile dropper bottles or unit dose vials.

### Example 4

### Preparing an Immunosuppressant-Based Pharmaceutical Composition For Use in the Treatment of Chronic Dry Eye Disease

A pharmaceutical composition was prepared as described below. The following products were used in the amounts specified:
(a) about 0.321 g of mycophenolate sodium powder;
(b) about 0.0255 g of tacrolimus monohydrate powder;
(c) about 0.25 g of chondroitin sulfate (bovine);
(d) about 0.25 of powdered dextran 70,000;
(e) about 0.1 g of edetate disodium powder;
(f) about 0.30 g of powdered sodium thiosulfate pentahydrate;
(g) about 0.20 g of PLURONIC F-127;
(h) about 1.0 mL polysorbate-80;
(i) about 4.0 mL polyethylene glycol 400 MW;
(j) about 1.0 mL of glycerol;
(k) about 1.17 g of sodium phosphate dibasic anhydrous;
(l) about 0.14 g of sodium phosphate monobasic anhydrous;
(m)about 0.10 g of METHOCEL E4M;
(n) about 40 mL of balanced salt solution; and
(o) about 100 mL of sterile injectable water.

Chondroitin sulfate, dextran, sodium thiosulfate, and PLURONIC F-127 were combined with the balanced salt solution and about 90% of the water and stirred until completely dissolved followed by addition of glycerol with continued stirring. The pH of the solution was then adjusted to about 7.0 using sodium hydroxide solution before introducing mycophenolate sodium.

With continued stirring, mycophenolate sodium and tacrolimus monohydrate were added slowly followed by adding METHOCEL E4M and adjusting pH to about 7.3-7.4 and the remainder of water was added. The solution was then filtered through a 0.2 micron filter into sterile dropper bottles or unit dose vials.

Although the invention has been described with the reference to the above examples, it will be understood that modifications and variations are encompassed within the spirit and scope of the invention. Accordingly, the invention is limited only by the following claims.

### Example 5

### Preparing a Corticosteroid-Based Pharmaceutical Composition For Use in the Treatment of Chronic Dry Eye Disease

A pharmaceutical composition was prepared as described below. The following components were used in the amounts specified:
(a) 0.01 g of betamethasone sodium phosphate powder;
(b) 0.25 g of chondroitin sulfate (bovine);
(c) 0.25 of powdered dextran 70,000;
(d) 0.20 g of PLURONIC F-127;
(e) 1.0 mL of glycerol;
(f) 1.17 g of sodium phosphate dibasic anhydrous;
(g) 0.4 g of sodium phosphate monobasic anhydrous;
(h) 0.10 g of METHOCEL E4M;
(i) 40 mL of balanced salt solution; and
(j) 100 mL of sterile injectable water.

Chondroitin sulfate, dextran, sodium thiosulfate, and PLURONIC F-127 were combined with 90% of the balanced salt solution and about 90% of the water and stirred until completely dissolved followed by addition of glycerol with continued stirring. The pH of the solution was then adjusted to about 7.0 using sodium hydroxide solution. A stock solution of 1% betamethasone was prepared by dissolving 1 gm of betamethasone sodium phosphate powder in 100 mL water and confirming the stock solution concentration by HPLC. 1ml of the 1% betamethasone stock solution was then added to the mixture with continued stirring.

With continued stirring, mycophenolate sodium was added slowly followed by addition of METHOCEL E4M, adjusting pH to about 7.3-7.4, and addition of the remainder of the water. The solution was then filtered through a 0.2 micron filter into sterile dropper bottles or unit dose vials.

### Example 6

### Preparing a Corticosteroid-Based Pharmaceutical Composition For Use in the Treatment of Episodic Flare Ups

A pharmaceutical composition was prepared as described below. The following components were used in the amounts specified:
(a) 0.02 g of betamethasone sodium phosphate powder;
(b) 0.25 g of chondroitin sulfate (bovine);
(c) 0.25 g of powdered dextran 70,000;
(d) 0.20 g of PLURONIC F-127;
(e) 1.0 mL of glycerol;
(f) 1.17 g of sodium phosphate dibasic anhydrous;
(g) 0.4 g of sodium phosphate monobasic anhydrous;
(h) 0.10 g of METHOCEL E4M;
(i) 40 mL of balanced salt solution; and
(j) 100 mL of sterile injectable water.

Chondroitin sulfate, dextran, sodium thiosulfate, and PLURONIC F-127 were combined with 90% of the balanced salt solution and about 90% of the water and stirred until completely dissolved followed by addition of glycerol with continued stirring. The pH of the solution was then adjusted to about 7.0 using sodium hydroxide solution. A stock solution of 1% betamethasone was prepared by dissolving 1 gm of betamethasone sodium phosphate powder in 100 mL water and confirming the stock solution concentration by HPLC. 2 ml of the 1% betamethasone stock solution was then added to the mixture with continued stirring.

With continued stirring, mycophenolate sodium was added slowly followed by addition of METHOCEL E4M, adjusting pH to about 7.3-7.4, and addition of the remainder of the water. The solution was then filtered through a 0.2 micron filter into sterile dropper bottles or unit dose vials.

The invention described herein may be embodied in other specific forms without departing from the spirit or essential characteristics thereof. The specific embodiments previously described are therefor to be considered as illustrative of, and not limiting, the scope of the invention.

What is claimed is:

1. A pharmaceutical kit comprising:
(a) a first composition comprising a corticosteroid, an immunosuppressant or both a corticosteroid and an immunosuppressant in a pharmaceutically acceptable carrier configured for administration to a subject’s eyes;
(b) a second composition comprising a corticosteroid in a pharmaceutically acceptable carrier configured for administration to the subject’s eyes, wherein the second composition has a higher concentration of corticosteroid than the first composition; and optionally
(c) a lubricating formulation configured for administration to the subject’s eyes that is pharmaceutical-free.

2. The kit of claim 1, wherein the first composition and second composition are each provided in a plurality of unit dose containers, wherein the kit has a different number of unit dose containers including the first composition compared to the second composition.

3. The kit of claim 1, wherein the first composition comprises the corticosteroid or the corticosteroid and the immunosuppressant, wherein the corticosteroid is selected from the group consisting of a betamethasone, betamethasone acetate, betamethasone sodium phosphate, loteprednol, a triamcinolone, triamcinolone acetonide, triamcinolone diacetate, triamcinolone benetonide, triamcinolone furetonide, triamcinolone hexacetonide, dexamethasone, fluorometholone, fluocinolone acetonide, prednisone, prednisolone, methylprednisone, corticol, cortisone, fluorocortisone, deoxycorticosterone acetate, aldosterone, and budesonide.

4. The kit of claim 1, wherein the first composition comprises the immunosuppressant or the corticosteroid and the immunosuppressant, wherein the immunosuppressant is selected from the group consisting of mycophenolic acid, tacrolimus, cyclosporine, or any pharmaceutically acceptable salt or derivative thereof.

5. The kit of claim 1, wherein the first composition comprises the immunosuppressant or the corticosteroid and the immunosuppressant, further wherein the immunosuppressant is selected from the group consisting of mycophenolate sodium, mycophenolate mofetil, and mycophenolic acid.

6. The kit of claim 1, wherein the first composition comprises the corticosteroid and the immunosuppressant, wherein:
the corticosteroid is selected from the group consisting of a betamethasone, betamethasone acetate, betamethasone sodium phosphate, loteprednol, triamcinolone, triamcinolone acetonide, triamcinolone diacetate, triamcinolone benetonide, triamcinolone furetonide, triamcinolone hexacetonide, dexamethasone, fluorometholone, fluocinolone acetonide, prednisone, prednisolone, methylprednisone, corticol, cortisone, fluorocortisone, deoxycorticosterone acetate, aldosterone, and budesonide; and
the immunosuppressant is selected from the group consisting of mycophenolic acid, mycophenolate sodium, mycophenolate mofetil, tacrolimus, and cyclosporine, or pharmaceutically acceptable salts or derivatives thereof.

7. The kit of claim 6, wherein the corticosteroid of the second composition is selected from the group consisting of a betamethasone, betamethasone acetate, betamethasone sodium phosphate, loteprednol, triamcinolone, triamcinolone acetonide, triamcinolone diacetate, triamcinolone benetonide, triamcinolone furetonide, triamcinolone hexacetonide, dexamethasone, fluorometholone, fluocinolone acetonide, prednisone, prednisolone, methylprednisone, corticol, cortisone, fluorocortisone, deoxycorticosterone acetate, aldosterone, and budesonide.

8. The kit of claim 7, wherein the corticosteroid of the first and second compositions is the same but at higher concentration in the second composition.

9. The kit of claim 1, wherein the kit comprises the lubricating formulation, wherein the lubricating formulation comprises Sodium Thiosulfate Pentahydrate at 0.3 %wt/wt; Sodium Chondroitin Sulfate at 0.25% wt/wt; Dextran-70 at 0.25% wt/wt; Edetate Disodium Dihydrate at 0.1 % wt/wt; Poloxamer 407 at 0.2% wt/wt; Glycerin at 0.2% wt/wt; Potassium Chloride at 0.03% wt/wt; Sodium Phosphate Dibasic Anhydrous at 0.90% wt/wt; Sodium Phosphate Monobasic Anhydrous at 0.18% wt/wt; Sodium hydroxide at Q.S. to adjust pH 7.2; Hydrochloric Acid, 37% at Q.S. to adjust pH 7.2; and Sterile Water Q.S. to 100%.

10. A method for the treatment of dry eye disease in a subject, comprising:
a) providing the kit of claim 1;
b) topically administering to the eyes of a subject suffering from chronic dry eye disease a therapeutically effective amount of the first composition over a treatment period; and if the subject suffers from an episodic flare up during the treatment period,
c) topically administering a therapeutically effective amount of the second composition to the eyes of the subject to substantially alleviate the episodic flare-up.

11. The method of claim 1, wherein after the episodic flare up is substantially alleviated, continuing to administer to the eyes of the subject the therapeutically effective amount of the first composition over the treatment period that remains.
